(19) United States
(12) Patent Application Publication
(10) Pub. No.: US 2019/0055515 A1
(43) Pub. Date: Feb. 21, 2019

(54) PRODUCTION OF SCHWANN CELLS

(71) Applicant: UNIVERSITY OF MIAMI, Miami, FL (US)

(72) Inventors: Dalton Dietrich, Miami, FL (US); Gagani Athauda, Miami, FL (US); Mary Bunge, Miami, FL (US); James Guest, Miami, FL (US); Allan Levi, Miami, FL (US); Damien Pearse, Miami, FL (US); Patrick Wood, Miami, FL (US)

(21) Appl. No.: 16/078,537

(22) PCT Filed: Feb. 24, 2017
(86) PCT No.: PCT/US17/19451

§ 371 Date: Aug. 21, 2018

Related U.S. Application Data
(60) Provisional application No. 62/299,726, filed on Feb. 25, 2016.

(51) Int. Cl. C12N 5/079; A61K 35/30; A61L 27/38

(52) U.S. Cl. CPC C12N 5/0622; A61K 35/30; C12N 2506/08; C12N 2533/52; C12N 2501/01; A61L 27/383

(57) ABSTRACT

The invention provides a method of producing a population of human Schwann cells. The method comprises (a) incubating human fascicles with one or more mitogens for a priming period of three to fourteen days to produce primed fascicles, (b) incubating the primed fascicles with one or more tissue dissociation enzymes to produce primed Schwann cells, (c) culturing the primed Schwann cells at an initial Po density for a period of time to achieve no greater than 90% confluence, (d) expanding the population of Schwann cells by culturing the Schwann cells at an initial passage density for a period of time to achieve no greater than 90% confluence for at least two passages, and harvesting the population of human Schwann cells. The invention further provides an isolated population of Schwann cells obtained by the method described herein. In various aspects of the invention, the isolated population is provided in a composition.

### CROSS REFERENCE TO RELATED APPLICATIONS

This application claims the benefit of priority to U.S. Provisional Patent Application No. 62/299,726, filed Feb. 25, 2016, the disclosure of which is hereby incorporated by reference in its entirety.

### STATEMENT OF GOVERNMENTAL SUPPORT

This invention was made with government support under NINDS 09923 awarded by the National Institutes of Health. The government has certain rights in the invention.

### TECHNICAL FIELD OF THE INVENTION

The disclosure relates to methods for producing a population of Schwann cells.

### BACKGROUND OF THE INVENTION

Within the field of regenerative medicine, the application of transplantation to repair injuries and to treat disease is accelerating. In terms of commercially developed cellular therapeutics, the majority of FDA-approved products such as umbilical cord blood derived hematopoietic stem cells are for hematological diseases. It is urgent that regenerative medicine be applied to injuries and diseases of the central nervous system (CNS) that lack reparative therapies. Spinal cord injury (SCI) is a CNS condition for which tissue repair and regeneration treatments are emerging. An estimated 265,000 people are living with chronic SCI, and there are 12,000 new injuries each year in the United States. When SCI caused by chronic spinal column degeneration is included, the number of persons affected is much greater.

Cellular transplantation has been proven to support axonal regeneration and myelin repair after SCI in several animal models. Axonal regeneration in peripheral nerves is dependent upon Schwann cells (SC). Efficient propagation of SC cultures suitable for transplantation has proven difficult and serves as a major obstacle to realizing the full potential of this regenerative therapy. Previously employed methods are plagued by limited SC yields that are unsuitable for large-scale manufacturing and, in some instances, require SC exposure to antibodies and other reagents that complicate the production of clinical products intended for human application.

### SUMMARY OF THE INVENTION

The invention includes, for example, a method of producing a population of human Schwann cells. The method comprises (a) incubating human fascicles with one or more mitogens for a priming period of three to fourteen days to produce primed fascicles, (b) incubating the primed fascicles with one or more tissue dissociation enzymes to produce primed Schwann cells, (c) culturing the primed Schwann cells at an initial Po density of 10,000 to 15,000 cells/cm² (e.g., 13,333 cells/cm²) for a period of time to achieve no greater than 90% confluence, (d) expanding the population of Schwann cells by culturing the Schwann cells at an initial passage density of 6667 cells/cm² to 13333 cells/cm² for a period of time to achieve no greater than 90% confluence for at least two passages, and harvesting the population of human Schwann cells.

In various embodiments, the method comprises (a) incubating human fascicles with forskolin and heregulin for eight days to produce primed Schwann cells, (b) incubating the primed Schwann cells with collagenase and neutral protease for 18 hours, (c) preparing a suspension of primed Schwann cells in laminin-coated tissue culture containers at an initial density of 10,000 to 15,000 cells/cm² (e.g., 13,333 cells/cm²), (d) culturing the Schwann cells until 60%-90% (e.g., 80%-90%) confluence, (e) passaging the Schwann cells into larger laminin-coated tissue culture containers at an initial passage density of 6667 cells/cm² to 13333 cells/cm², (f) passaging the Schwann cells when 60%-90% (e.g., 80%-90%) confluence is obtained no more than three times, wherein the Schwann cells are seeded at an initial passage density of 6667 cells/cm² to 13333 cells/cm² at each passage, (g) harvesting the population of human Schwann cells, and (h) washing the harvested population of human Schwann cells at least twice.

The invention further provides a population of Schwann cells obtained (or obtainable) by the method described herein.

### DESCRIPTION OF THE FIGURES

FIG. 1 is a diagram illustrating an embodiment of the invention. A sketch of the nervous system is provided. First, a segment of peripheral nerve, e.g., the sural nerve, is surgically harvested. Second, the nerve is dissected to extract the fascicles to separate the SC-containing nerve portions from other tissues of the nerve. Third, the nerve fascicles are primed in cell culture for a period of time, e.g., eight days. During this stage, in various embodiments, the SC begin to separate from axons and myelin. Exposure to mitogens accelerates SC dedifferentiation. Fourth, following enzymatic dissociation of the nerve fascicles, the cells are seeded at a relatively low density as a monolayer on laminin-coated plastic. Fifth, multiple cell divisions result in a more confluent cell monolayer, and the cells are passaged into multiple, larger monolayer surfaces for the second passage. Sixth, upon reaching a desired confluence, the cells are harvested for transplantation.

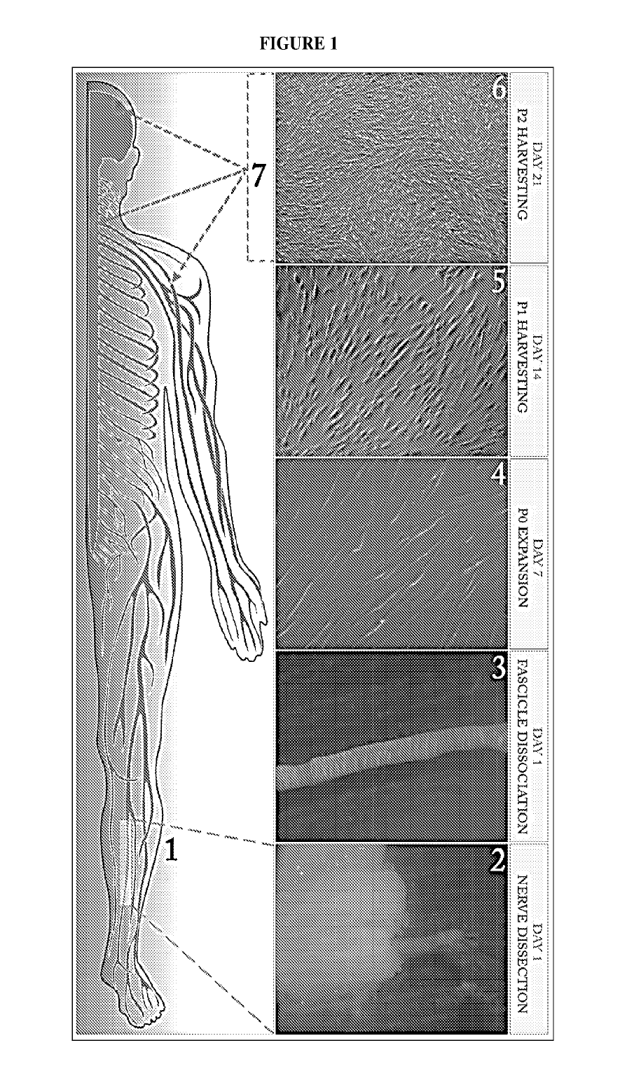

FIG. 2 is an illustration of an exemplary method for producing a population of purified SC. 1. A nerve consists of many axons organized in bundles and enclosed in fascicular structures. SC are associated with axons; they may be myelinating one single axon or ensheathing several smaller caliber axons. The human sural nerve may have seven or more fascicles, each protected by a perineurial layer. The fascicles are pulled away from fibrous sheaths using, e.g., dissection microscopy techniques. 2. The fascicles are cut into shorter lengths and placed in media with mitogens to prime the fascicles. 3. During priming, axonal fragmentation and the separation of SC from myelin begins, and the SC commence dedifferentiation. If present, heregulin and forskolin initiate signaling pathways to enhance SC division after fascicle dissociation and plating (Po). 4. The product is enzymatically dissociated, and the cellular pellet containing the different cellular types in the nerve is plated at a density of, e.g., 1×10⁶ on T-75 laminin-coated flasks. This is referred to as the P0 stage. Expansion in P0 is continued until, e.g., 60% to 90% (e.g., 80% to 90%) confluence is reached (allowing low cell-cell contact and rapid division). The monolayers are released from these flasks (P1-passage 1) and seeded to, e.g., CellStack chambers (636 cm²) for the final expansion. In various aspects, the product of this final expansion is harvested for transplantation at P2 (passage 2).

### DETAILED DESCRIPTION OF THE INVENTION

The invention provides, in various aspects, a manufacturing process suitable for producing human SC for use in human patients on an industrial scale. Using previous methods, it was difficult to achieve sufficient quantities of SC suitable for human use. For example, SC are prone to growth arrest in cell culture despite continued mitogen exposure. Many previous methods require exposure to multiple reagents not suitable for human use that complicated purification. Additionally, existing methods used to cultivate rodent SC are not consistently suitable for producing human SC, as rodent SC may undergo many more culture passages than human SC before growth arrest, making methods suitable for non-human cells unpredictable for human SCs.

Indeed, the challenges to systematically produce human SC therapeutic cultures are substantial. The number of SC required for the clinical application is many fold greater than the original number of SC that can be derived from the nerve biopsy, making expansion essential. The original SCs must be derived from a complex nerve tissue containing blood vessels, connective tissue compartments, fat, and nerve fascicles. The SCs within a freshly harvested nerve are in a differentiated state and many are forming dense myelin wraps around axons. To selectively manufacture SC, the nerve must be deconstructed and the SCs dedifferentiated to allow division as a two-dimensional monolayer. The promotion of cell division must not irreversibly alter the genetic program of the cells to create a risk of neoplasia after transplantation. The culture must be cleared of the devitalized axon fragments, associated myelin, and debris to avoid excess inflammation after implantation. Some constituent cells of the nerve, such as fibroblasts, are more adherent and likely to divide in monolayer cell culture than SCs. Such cells preferably are suppressed or selectively eliminated (in whole or in part) to avoid or reduce contamination. Furthermore, the resulting SC population, in suspension, preferably exhibit fluid properties amenable to injection through clinically suitable devices. These properties include, but are not limited to, tolerance to the shear stress of injection through needles or tubing, and the ability to return to a uniform concentration with minimal clumping by mild mechanical agitation after cell settling.

The application provides materials and methods for manufacturing clinically suitable SC products wherein, in various embodiments, source nerve tissue is deconstructed, SC are dedifferentiated, and SC are efficiently expanded with increasing purity to generate a population of potent, viable cells. The method described herein produces SC populations within a clinically suitable timeframe and in a quantity required for therapeutic application, while maintaining a low passage number. The resulting SC cells exhibit multiple reparative effects following transplantation, and are suitable for, e.g., delivery to spatially complex regions of CNS injury, combination with biomaterials for sustained release or localized deposit into a region of the body, or combination with nerve grafts to enhance peripheral nerve repair.

In one aspect, the application provides a method of producing a population of human Schwann cells (SCs). The method comprises (a) incubating human fascicles with one or more mitogens for a priming period of three to fourteen days to produce primed fascicles, (b) incubating the primed fascicles with one or more tissue dissociation enzymes to produce primed Schwann cells, (c) culturing the primed Schwann cells at an initial P0 density of 10,000 to 15,000 cells/cm² (e.g., 10,000 to 13,333 cells/cm², such as 13,333 cells/cm²) for a period of time to achieve no greater than 90% confluence, (d) expanding the population of Schwann cells by culturing the Schwann cells at an initial passage density of 6667 cells/cm² to 13333 cells/cm² for a period of time to achieve no greater than 90% confluence for at least two passages, and (e) harvesting the population of human Schwann cells.

In various aspects of the invention, the human fascicles are extracted from human nerve tissue following dissection. The donor nerve is surgically harvested under sterile technique. If transported, the tissue is placed in a sterile specimen cup containing Belzer's (Viaspan UW solution) transport media and maintained between 2-10 degrees Centigrade. While the method is not dependent on the particular amount of starting material, optionally the nerve length of the sample is about 15 cm or longer. Optionally, the human fascicles are isolated from human sural nerve tissue, although other peripheral nerves also can be used in the context of the invention.

In some embodiments, the human fascicles are extracted from human nerve tissue one day or more following dissection, e.g., two or more, three or more four or more, five or more, six or more, seven or more, eight or more, nine or more, or ten or more days. Alternatively, the human fascicles are extracted from human nerve tissue no more than 14 days following dissection, e.g., no more than 12, no more than 10, no more than nine, no more than eight, no more than seven, no more than six, no more than five, no more than four, or no more than three days following the dissection. In various embodiments, the human fascicles are extracted from human nerve tissue no more than three days following the dissection. Optionally, the human fascicles are extracted from human nerve tissue about seven days following dissection. In this regard, the tissue is preferably stored at 4° C. Fascicles also may be extracted the day of harvest.

Extraction of fascicles from nerve tissue is performed using any suitable technique. For example, in one embodiment, the nerve is placed in a plastic dish containing, e.g., cold Leibovitz L-15 containing gentimicin 50 μg/ml, under a dissecting microscope and non-neural connective tissue is extracted. Using delicate tipped forceps, the endoneurial nerve fascicles are pulled individually away from the surrounding perineurium and cut into, e.g., 1 cm long explants. Optionally, the volume of extracted fascicles is measured and recorded.

The dissected fascicles are incubated with one or more mitogens for a priming period of three to fourteen days to produce primed fascicles. As described herein, the priming step allows the SC cells to dedifferentiate and also enhances division. Suitable mitogens include, for example, forskolin, heregulin, and a combination of forskolin and heregulin. Other SC mitogens include, e.g., pituitary extract and cholera toxin. The fascicles are incubated in any cell culture medium suitable for maintaining SC. Examples of medium include, but are not limited to, DMEM F12 and RPMI. In this regard, the fascicles optionally are incubated in a SC growth media comprising DMEM F12 supplemented with 4 mM glutamine and comprising forskolin (2 μm) and heregulin B1 (10 nm). The media used for priming and/or expansion (P0, P1, P2, P3, etc.) contains serum (e.g., fetal bovine serum) in various embodiments. In one aspect, the fascicles are placed in uncoated flasks (e.g., T-75 uncoated flasks) with fresh SC growth media (e.g., 8 mL) and placed inside an incubator at, e.g., 8% CO₂ at 37° C. The priming period is three to fourteen days, e.g., three to twelve days, three to ten days, three to eight days, five to fourteen days, five to ten days, or five to eight days. Optionally, the priming period is seven or eight days in length. During the priming stage, the SC growth media may be changed as needed, e.g., every two days.

The method further comprises incubating the primed fascicles with one or more tissue dissociation enzymes to produce primed Schwann cells. The tissue dissociation enzyme(s) preferably digests connective tissue components, such as collagen and fibronectin. In various aspects of the invention, the tissue dissociation enzyme is a metalloprotease. Suitable metalloproteases include, but are not limited to, collagenase, neutral protease, and a combination of collagenase and neutral protease.

The amount of tissue dissociation enzyme employed to produce primed Schwann cells will vary with the amount of fascicles, and those of skill in the art can determine the quantity of enzyme using routine methods. One means of determining the quantity of enzyme for use in a particular embodiment of the invention is based on “packed fascicle volume.” For example, after the priming period (e.g., on day eight of culture), the primed fascicles are placed into a 15 cc centrifugation tube and spun at 20×g for one minute to determine the “packed fascicle volume.” The volume of enzyme mixture added is equivalent to the packed fascicle volume×7 mL/0.3 mL (e.g., 7 mL of dissociation enzyme solution is needed for every 0.3 mL of fascicle pellet volume). In one embodiment, the one or more tissue dissociation enzymes (e.g., collagenase NB1 and neutral protease NB (Serva electrophoresis, GMP grade, Germany)) is provided in a mixture of DMEM with high glucose, 3.1 mM CaCl₂. The fascicles are optionally incubated with the tissue dissociation enzyme(s) at 37° C. with 8% CO₂ for a period of time sufficient to produce primed Schwann cells. For example, in various embodiments, the primed fascicles are incubated with the tissue dissociation enzyme for 12 hours to 24 hours (e.g., 12 hours to 18 hours, 16 hours to 18 hours, or 16 hours to 24 hours). In one aspect, the primed fascicles are incubated with the tissue dissociation enzyme for 18 hours.

Following incubation, the dissociation enzyme(s) may be neutralized. An exemplary method for neutralizing the tissue dissociation enzymes includes adding a volume of fetal bovine serum (FBS) that is 10% of the volume of dissociation enzyme(s) used. The fascicles are dissociated by, e.g., mechanical disruption, such as by gently pipetting up and down several times. Tissue dissociation enzymes and residual cellular debris is then preferably removed. In this regard, one embodiment of the invention comprises transferring the dissociate to a 50 cc conical tube, adding D-10 culture media until the total volume is 40 cc, and centrifuging the tube at 150×g for five minutes at 4° C. The washing process is optionally repeated two additional times, although fewer (e.g., one) or more (e.g., three, four, or five) washes also are contemplated.

The resulting primed SCs are reconstituted in cell culture media (e.g., SC medium described herein), which is optionally supplemented with antibiotic, such as gentamycin. In various embodiments, the primed SC are reconstituted at a concentration of 1×10⁶ cells/mL. The primed SC are plated, preferably in a container comprising a laminin-coated surface. Laminin is an extracellular matrix molecule produced by SC, which in various aspects, adsorbs to tissue culture plastic and provides an excellent adhesion substrate for SC. One suitable container for initial expansion of the primed SC is a T 75 cm² flask pre-coated with mouse laminin. An exemplary method for pre-coating the flasks includes adding 75 μl of 1 mg/ml laminin solution to 10 cc of DPBS and applying the mixture to a substrate in a 37° C., 8% CO₂ humidified incubator for two hours, then rinsing the substrate with, e.g., three washes of 10 cc of DPBS.

The resulting primed SC are cultured at an initial density (“P0”) of about 10,000-15,000 cells/cm² (e.g., 10,000-13,333 cells/cm², such as 13,333 cells/cm²) for a period of time to achieve no greater than 90% confluence. The initial density provides an ideal environment for maximal cell expansion that minimizes the growth of contaminating cells, e.g., fibroblasts. P0 density is greater than that used for subsequent passages (P1, P2, P3, etc.). In one embodiment, initiation of monolayer SC expansion comprises seeding 1×10⁶ cells per T 75 cm² flask by combining 1 cc of cells at 1×10⁶ cells/ml with 9 cc of SC growth medium. The flasks containing the cells are incubated at 37° C. with 8% CO₂. Generally, the cell culture media is changed every two days, although alternative timing also is contemplated in the context of the inventive method. Changing the media every two days refreshes the concentration of growth factors, provides energy substrates, and maintains stable pH while permitting a medium conditioning by factors released by the SC.

The initial expansion is allowed to proceed for a period of time to achieve no greater than 90% confluence, e.g., 50%-90% confluence, 60%-90% confluence, 60%-80% confluence, 70%-90% confluence, 80%-90% confluence, or 60%-70% confluence, and then passaged. Preferably, the SC are cultured for a period of time to achieve 60%-90% (e.g., 80%-90%) confluence. While not wishing to be bound by any particular theory, SC cells at 90% confluence or less (e.g., 60-80% confluence) remain in the exponential growth phase, which maximizes ultimate yield and purity of the final SC product when the first passage is performed at this timepoint. Confluence is determined by any suitable method, such as image analysis of sampled regions of the flasks.

The method described herein also promotes a continual increase in the fraction of SC in the culture (i.e., the proportion of SC in the culture increases compared to the proportion of contamination cells (e.g., fibroblasts)). In various embodiments, directly following the P0 expansion, the method comprises determining the percentage of Schwann cells in the culture and, if the percentage is less than 80%, culturing the Schwann cells in an uncoated container (e.g., a container lacking laminin) for a period of time to allow fibroblast adhesion to the container. In this optional step, use of an uncoated container promotes differential adhesion of fibroblasts that rapidly adhere to the plastic surface, thereby substantially enriching the population for SC. For example, the SC culture is incubated in the uncoated container for a period of time to allow at least 10%, at least 20%, at least 30%, at least 40%, at least 50%, at least 60%, at least 70%, at least 80%, or at least 90% (e.g., 10%-99%, 50%-90%, 50%-70%, or 70%-90%) of contaminating fibroblasts to adhere to the container surface and be removed from the SC culture. In various embodiments, the SC culture is incubated in the uncoated container for 5-60 minutes, e.g., 10-30 minutes, 10-20 minutes, 5-15 minutes, or 5-10 minutes.

The method of the disclosure further comprises expanding the SC culture by culturing the SC at an initial passage density of 6667 cells/cm² to 13333 cells/cm² for a period of time to achieve no greater than 90% confluence. To illustrate, SC expansion following the initial plating is achieved by seeding the cells released from the T 75 flasks following P0 onto a larger cell culture container than that used for the P0 expansion, the cell culture container optionally having a laminin-coated surface. Exemplary P1, P2, P3, etc. cell culture containers include 636 cm² cell culture chambers, which provide an 8.5× increase in monolayer surface area over T 75 cm² flasks. The media employed typically does not comprise antibiotics, which avoids the risk that a cell culture infection is masked by a bacteriostatic effect, possibly leading to implantation of an infected cell product. The SC medium is optionally replenished every two days until harvest.

The expansion stage is performed for at least two passages (e.g., two passages, three passages, or four passages). In various embodiments, the expansion stage includes no more than five passages, no more than four passages, or no more than three passages. During each passage, the cells are cultured for a period of time to achieve no greater than 90% confluence, e.g., 50%-80% confluence, 60%-70% confluence, 60%-80% confluence, 60%-90% confluence, 70%-80% confluence, 70%-90% confluence, or 80%-90% confluence, and then passaged. Preferably, the SC are cultured for a period of time to achieve 60%-90% (e.g., 80%-90%) confluence during each passage.

In various embodiments, the expansion stage comprises two passages. For most SC products, 60%-90% (e.g., 80%-90%) confluence within two or three 636 cm² flasks will provide adequate cells for transplantation. Typically, obtaining sufficient cells for a final SC product suitable for transplantation occurs in about three weeks following nerve harvest, but it may be earlier or later for individual preparations. In one embodiment of the invention, the method further comprises washing the population of Schwann cells at least two times.

An example of a method for harvesting the SC begins with aspirating the cell culture medium from the flask and replacing it with 50 cc of Ca2⁺/Mg²⁺ free Hank's Balanced Salt Solution (HBSS), which is, in turn, aspirated and replaced with 20 cc of 1× TrypLE select. Trypsinization proceeds for 10 minutes at room temperature and is stopped by the addition of 47 ml of culture medium to each flask (wash step 1). The released cell suspension is transferred to 50 cc conical centrifuge tubes that are centrifuged at 150×g for five minutes at 4° C. The medium is aspirated, leaving the cell pellet undisturbed, and 5 cc of DMEM/F12 is added to resuspend the cells (first resuspension). The 5 cc quantities are pooled into a single 50 cc conical tube, and the total volume is increased to 40 cc by adding DMEM/F12 (wash step 2). The tube is centrifuged (150×g for five minutes at 4° C.), and the cells again resuspended in a total of 40 cc DMEM/F12 (wash step 3). Optionally, samples are taken and tested for viability; anaerobic, aerobic and fungal testing; and/or endotoxin testing. The supernatant from wash step 3 is removed, the cells are resuspended in 1 cc of DMEM/F12 and again centrifuged. An amount of supernatant is removed to produce a final cell concentration of 10,000 to 200,000 cells/μl (e.g., 50,000 to 100,000 cells/μl, such as 100,000 cells/μl). These P2 human SC are the final product for transplantation. The methodology described above is merely one embodiment of the inventive method provided for the purposes of illustration.

The invention further provides a method of producing a population of human Schwann cells, the method comprising (a) incubating human fascicles with forskolin and heregulin for eight days to produce primed Schwann cells, (b) incubating the primed Schwann cells with collagenase and neutral protease for 18 hours, (c) preparing a suspension of primed Schwann cells in laminin-coated tissue culture containers at a density of 13,333 cells/cm², (d) culturing the Schwann cells until 60%-80% confluence, (e) passaging the Schwann cells into larger laminin-coated tissue culture containers at an initial passage density of 6667 cells/cm² to 13333 cells/cm², (f) passaging the Schwann cells when 60%-90% (e.g., 80%-90%) confluence is obtained no more than three times, wherein the Schwann cells are seeded at an initial passage density of 6667 cells/cm² to 13333 cells/cm² at each passage, (g) harvesting the population of human Schwann cells, and (h) washing the harvested population of human Schwann cells at least twice.

The invention further provides an isolated population of Schwann cells obtained (or obtainable) by the method described herein. In various aspects of the invention, the isolated population is provided in a composition that maintains substantially consistent viscosity, maintains two phase suspension, allows movement of air bubbles, provides a high cell concentration, and/or tolerates shear stress.

The method described herein maximizes the viability and stability of the SC product, while reducing the concentration of mitogens, allogeneic proteins and peptides, and excipients that are largely unsuitable for cell transplantation products. The number of SC cells produced is sufficient to treat substantial injury with a high density suspension exceeding e.g., 50 million cells. The SC purity of the isolated population produced by the method described herein optionally exceeds 80%, 85%, 90% 95%, or 98%. The purity (or percentage of SC in the isolated population compared to other cell types) is determined by any suitable means. For example, purity is demonstrated by expression of surface marker CD271 in flow cytometry assay, and by detection of S-100 immunostaining in fixed cells as compared to detection of fibronectin-positive fibroblasts. Further, the level of excipients remaining in the final cell product is typically below the level of detection or is so low as to have no meaningful biological activity.

Additionally, in various aspects, the viability of the population of SC is greater than 80%, 85%, 90%, or 95% at eight hours after final preparation. In some aspects, viability is greater than 80%, 85%, 90%, or 95% at 12 hours, 18 hours, or 24 hours following harvest. Viability is characterized by any of a number of methods, such as by labeling living and dead cells with SYTO 24 and SYTOX Green, respectively, and performing automated cell counting using a Cellometer Vision (Nexcelom Bioscience) cell counter. The enhanced stability of the resulting population of SC is an unexpected technical advantage, and allows sufficient time to transfer the population of cells to an operating facility for transplantation.

The invention further provides a method for treating a nerve injury comprising administering to a subject in need thereof (e.g., a human) a population of Schwann cells obtained (or obtainable) by the method described herein. The Schwann cells may be derived from the subject (i.e., autologous) or derived from a different subject (e.g., heterologous). In various embodiments, the population of Schwann cells is administered directly to the injury site. In various aspects, the injury is a peripheral nerve (e.g., sciatic nerve) injury, a central nerve (e.g., spinal cord) injury, or both. Nerve injury can occur in any of a number of contexts, including laceration, focal contusion, stretch injury, infection, and the like. Methods of diagnosing and characterizing the severity of nerve injury are known in the art and include, for example electromyogram (EMG) and nerve conduction study, CT scan, and MRI. See also e.g., Goubier et al. (2015). Nerves and Nerve Injuries, Vol. 2, Chapter 38, Elsevier Ltd, retrieved from www.drjngoubier.com/app/download/21998794/Grading+Nerve+Injuries.pdf. The amount administered optionally ranges from about 20 million cells to 200 million cells. As used herein, “treating” and “treatment” refers to any reduction in the severity and/or onset of symptoms associated with nerve injury. In various embodiments, administration of the population of Schwann cells results in restoration of motor function and/or strength (in whole or in part), sensory restoration (in whole or in part) (e.g., recovery of pinprick, slight touch, vibration, or heat/cold sensations), or both.

### EXAMPLES

### Example 1

This Example describes the treatment of two long-segment (7.5- and 5-cm), sciatic nerve injuries where SCs were combined with an autologous nerve construct. These represent the first two cases in which autologous SCs were transplanted into peripheral nerve injuries in humans.

Case 1:

The patient was a 25-year-old woman who sustained multiple lacerating injuries to her left lower extremity due to a boat propeller accident. This caused extensive damage to the left thigh and leg, and the patient was taken emergently to the operating room for control of vascular injuries and debridement of tissue. At the time of initial exploration, complete transection of the sciatic nerve was noted and the damaged nerve ends were sutured to adjacent muscle to prevent retraction. A small (0.5-cm) segment of the already damaged sciatic nerve stump was taken for SC harvest and propagation. Three days later the patient underwent repair of her lacerated Achilles tendon, during which a 5-cm segment of sural nerve was taken for autologous SC preparation. The sural nerve had also been previously injured, along with the tendon, by the propeller blade. Prior to sciatic nerve repair and SC transplantation, the patient also underwent anterior quadriceps washout and tendon repair as well as skin grafting of the posterior thigh. The patient ultimately underwent sciatic nerve repair and SC transplantation 30 days after injury and SC harvest.

Case 2:

The patient was a 30-year-old woman who suffered a gunshot wound to the posterior right midthigh that resulted in bullet fragments being lodged within the sciatic nerve. No initial surgical debridement of the wound was performed, but the patient underwent sural nerve biopsy in which a 5-cm graft was obtained for SC culture. Sciatic nerve repair with autologous SC transplant was performed 29 days after sural nerve harvest of SCs.

Human SC Harvesting and Cell Culture:

Autologous SCs were harvested from a small (0.5-cm) piece of traumatized sciatic nerve at initial debridement and from a 5-cm sural nerve biopsy conducted 3 days after the initial trauma in Case 1. All SCs in Case 2 were harvested from a 5-cm sural nerve biopsy conducted 9 days postinjury. Sural nerve biopsies for both patients were done on the side ipsilateral to the injury. Samples were placed in a Belzer solution, refrigerated at 4° C., and transported to the cell-manufacturing laboratory (current Good Manufacturing Practices [cGMP] facility) for cell culture. Sural nerve and sciatic nerve biopsies were dissected, and fascicles were pulled from epineurium and transferred to a triangular T-75 flask (Corning). The flask was placed in an incubator at 37° C. with 8% CO₂.

Culture medium, containing 1×DMEM (Life Technologies), 10% fetal bovine serum (Hyclone, GE Healthcare Life Sciences), 2 mM forskolin (Sigma-Aldrich), 10 nM human recombinant heregulin β1 (Genentech), 4 mM L-glutamine (Life Technologies), and 0.064 mg/ml gentamicin (APP Pharmaceutical/Fresenius Kabi USA), was changed every other day. On Day 7 for sciatic and Day 5 for sural nerves, the dissociation enzyme solution (5 ml) that contained neutral protease NB (2 dimethyl-casein units (DMCU)/ml; SERVA Electrophoresis GmbH), collagenase NB 1 (0.5 (PZU)/ml; SERVA Electrophoresis GmbH) in 1× high-glucose DMEM (Life Technologies) supplemented with 3.1 mM CaCl₂ (International Medication Systems Limited) was added to the fascicles and placed inside the incubator at 37° C. with 8% CO₂ for 18 hours. The fascicles were dissociated, 10 ml D-10 (Life Technologies) was added to the flask containing the fascicles, and it was centrifuged at 150 g for 5 minutes at 4° C. to pellet the cells. The cells were then washed two more times and plated onto mouse laminin-coated plates (1 μl/cm2, with a stock concentration of 1 mg/ml; Sigma-Aldrich) using the culture medium. The cells were fed with culture medium every three days. After seven days, cells reached 80% confluence for the nerve preparations.

For the samples obtained in Case 1, the viable cell count of sural nerve was 19.2 million cells and of the sciatic nerve the count was 10 million cells. The SC purity assessed by immune staining for sural nerve was 90.2% and for sciatic nerve it was 97%. For samples obtained in Case 2, the viable cell count of sural nerve was 270 million cells. The SC purity assessed by immune staining for sural nerve was 98.7%. The final cell products were washed three times to remove mitogens, laminin, and bovine products. Several controls were used throughout the manufacturing process to ensure that the product was essentially free of process-related contaminants. These controls included the wash steps described above and release testing of the final product. Investigations into the potential related impurities of the manufacturing process had been conducted during process validation studies at the time of our Investigational New Drug submission. The samples were analyzed for residual levels of heregulin b1 peptide, mouse laminin, gentamicin, and bovine serum albumin.

The total SC count was 28.8 million at a concentration of 100,000 cells/μl with >99.9% viability for Case 1, and 180 million at a concentration of 100,000 cells/μl with 97.8% viability for Case 2. Cells were placed on ice and transported to the operating room for transplantation.

Method of Transplantation:

In Case 1, repair of the sciatic nerve with SC transplantation took place 30 days postinjury. Complete transection of the sciatic nerve was noted at exposure. After debridement of scarred nerve ends, the sciatic nerve defect measured 7.5 cm. Bilateral sural nerves were harvested, and 12×7.5-cm nerve grafts were placed and then sutured using 7-0 prolene. A total of 28.8 million autologous SCs were supplemented within a Duragen Secure Dural Regeneration Matrix (Duragen; Integra LifeSciences Corp.).

In Case 2, sciatic nerve repair took place 41 days after initial injury and 29 days after sural nerve harvest of SCs. Two bullet fragments were found embedded within the sciatic nerve upon exposure. The bullet fragments were removed from the nerve using microsurgical technique. The tibial and peroneal nerve divisions were separated and intraoperative nerve action potential and ultrasound studies were performed. Results of the nerve action potential and ultrasound studies demonstrated an intact and functioning peroneal nerve and obvious damage to one-third of the tibial component. Scar tissue was removed and the tibial component was repaired. After removal of scarred nerve ends, the tibial nerve defect measured 5 cm. Sural nerve was obtained and 3×5-cm nerve grafts were placed and then sutured using 7-0 prolene. A total of 110 million autologous SCs of the original 180 million SCs were supplemented within a Duragen Secure Dural Regeneration Matrix.

Postoperative Follow-Up:

Lengths of follow-up were 36 and 12 months for the patients in Cases 1 and 2, respectively. Patients were serially tested for motor and sensory function according to the Medical Research Council (MRC) grading scale.

### Case Reports

Case 1:

At the time of injury and preoperatively, the patient had complete sensory loss to pinprick and light touch without allodynia in the distribution of the sciatic nerve. Motor function was completely lost below the knee (MRC Grade 0/5), hip flexion and knee flexion contracted against gravity (MRC Grade 3/5), and knee extension was active against resistance (MRC Grade 4/5). Pain was maximal postinjury (DN4 questionnaire; 10/10) in the distribution of the sciatic nerve. This pattern was consistent with a complete transection of the sciatic nerve at the upper thigh.

There were no postoperative complications with the nerve harvest and sciatic nerve repair within the posterior thigh. The patient did undergo debridement and antibiotic therapy of the anterior thigh for a methicillin-sensitive Staphylococcus aureus infection after a quadriceps tendon repair. She continued daily exercises and physical therapy post-repair. The patient had neurological assessments at 3-month intervals as well as MRI and ultrasound imaging postoperatively at 6, 12, and 30 months.

Over the course of 36 months the patient's neurological examination results gradually improved. At 15 months there was slight recovery of pinprick and light touch sensation in the distribution of the superficial peroneal nerve, which remains stable at 36 months. No sensation was recovered in the distribution of the sural, deep peroneal, and medial calcaneal nerves. Motor recovery of foot plantar flexion was first noted at 15 months, and at 30 months she achieved full strength recovery (MRC Grade 5/5), which is a definitive sign of regeneration across the sural nerve and autologous SC construct within the tibial division of the sciatic nerve. By the 36-month follow-up, on contraction with gravity eliminated (MRC Grade 2/5) in foot dorsiflexion and foot eversion, but had no recovery (MRC Grade 0/5) in toe dorsiflexion and foot inversion. Pain gradually diminished over time, and at her 36-month follow-up she was 0/10 without pain medication and after weaning from gabapentin. She does report occasional lancinating pain in the distribution of the sciatic nerve. Postoperative MRI and ultrasound studies demonstrated continuity of the grafts to both sciatic nerve ends, with no tumor formation. Of note, the patient completed a 5-k run around the time of her 36-month follow-up.

Case 2:

At the time of injury, the patient had no sensation in the sciatic distribution of the right lower extremity. Forty-one days later (preoperatively), the patient had diminished sensation to light touch and pinprick in the sural and superficial peroneal distribution, diminished light touch and no pinprick sensation in the medial calcaneal distribution, and no sensation in the deep peroneal distribution. Preoperatively, motor function was absent (MRC Grade 0/5) in foot eversion and inversion; trace contraction (MRC Grade 1/5) was seen in toe dorsiflexion, foot dorsiflexion, and plantar flexion; knee flexion was contracted against gravity (MRC Grade 3/5); and knee and hip flexion were at full strength (MRC Grade 5/5). Postinjury, the patient noted DN4 questionnaire 10/10 sharp pain in the distribution of the sciatic nerve, which improved to DN4 questionnaire 8/10 immediately prior to the nerve repair. Examination and imaging findings were consistent with damage to the sciatic nerve at the midthigh.

There were no postoperative complications with the sciatic nerve repair and SC transplantation. The patient had neurological assessments at 3-month intervals as well as MRI at 4 months and ultrasound at 12 months.

Over the course of 12 months the results of the patient's neurological examination gradually improved. At 12-month follow-up there was significant improvement in toe dorsiflexion (MRC Grade 4/5), foot plantar flexion (MRC Grade 5/5), and inversion (MRC Grade 4/5). Complete restoration in motor strength demonstrates the efficacy of sciatic nerve repair with supplementation of SCs. Pain gradually improved over the course of follow-up visits in both distribution and intensity. At 3 months the distribution only involved the lateral leg and foot, and subsequently only the foot at 12-month follow-up. Intensity decreased to DN4 questionnaire 3/10 at her 12-month follow-up visit. The MRI and ultrasound studies demonstrated continuity of the grafts to both sciatic nerve ends, with no tumor formation.

### Discussion

Sciatic nerve injuries are relatively rare, yet they are some of the most challenging cases a peripheral nerve surgeon will face. Damage to the sciatic nerve can occur through a variety of means. Iatrogenic causes such as intragluteal injections and hip joint repair, as well as hip fractures or dislocations and penetrating trauma commonly injure the upper sciatic nerve. Stab wounds, gunshot wounds, and boat propeller injuries are commonly associated with midsciatic injury. Injury location and sciatic division have been associated with differing rates of success after nerve autograft repair. High sciatic injuries involving the peroneal component have been associated with poor outcomes, whereas midthigh injuries to the tibial component have had higher rates of success. Along with location and sciatic division, nerve gaps >5 cm have been associated with worse outcomes.

Repair of a damaged sciatic nerve that has a significant gap is particularly challenging due to several factors. Sural nerves, the most common donor autograft, are typically insufficient in length due to the large discrepancy in cross-sectional area between donor and sciatic nerve. Patients with thin sural nerves may only be able to cover a 2.5-cm gap. In addition to insufficient graft material, sensory loss at the donor site and possible neuroma formation are possible morbidities associated with autologous sensory nerve graft transplants.

Autologous SCs can be harvested from either a donor nerve or from the epicenter of the traumatized nerve ends in sharp injuries (propeller injury, gunshot wound, stab wound). Harvesting SCs from donor nerves requires sacrifice of sensory donor nerves, which may lead to future morbidity, whereas harvesting from the traumatized nerve ends will lead to no deficit because these ends will eventually scar and be sacrificed by the surgeon. In this study, SCs from the patient in Case 1 were harvested from both a donor nerve and the traumatized nerve, whereas SCs from the patient in Case 2 were only harvested from a donor sural nerve. Both methods provided sufficient samples to propagate SCs in culture until the time of surgery (30 days for Case 1 and 29 days for Case 2).

Presented here are the first two cases treated using autologous SCs to supplement human peripheral nerve repair in long-segment injury. Both patients had significant improvement in both motor and sensory function, with correlative imaging, after large-gap (>5 cm) injuries generally associated with poor functional recovery. Near complete resolution and significant improvement in pain symptoms in the patients in Cases 1 and 2, respectively, were observed—an outcome rarely seen in autologous nerve grafting. A proposed mechanism for pain reduction in entubulation strategies is that AGCs provide a scaffold for more directional growth of axons and less growth of pain fibers. Donor SCs were able to propagate to sufficient amounts for transplantation in both patients far earlier than 4 months, when poor outcomes are seen. Grafts remained in continuity and no neuromas or tumors were seen at 12 and 36 months.

### Example 2

This Example describes transplantation of human SCs (HuSCs) into a contusive SCI paradigm in the nude rat (Kreutziger et al., Tissue Eng Part A 17:1219-28 (2011); Numasawa et al., Stem Cells, 29:1405-14 (2011)). The cells were used as either fresh isolates or from cryopreserved preparations, compatible with cellular storage. The evaluation of safety included assessments of HuSC persistence, proliferation, tumorgenicity and biodistribution along the neural axis from 3 days to 6 months post-transplantation. Measurement of host responses to the presence of HuSCs involved the analysis of immune cell infiltration, glial scar formation, tissue preservation and neuropathology. Lastly, the ability of HuSCs to support axon growth and myelination was examined.

### Materials and Methods

Preparation of HuSC Cultures:

HuSCs were obtained independently from six cadaveric and organ donor sural nerve biopsies following methods previously described by Casella and colleagues (Casella et al., Glia, 17:327-38 (1996)) with a number of modifications to achieve large numbers of highly pure HuSCs for clinical application (Levi et al., Cell Transplant, 25:1395-403 (2015)). Sural nerve biopsies were processed under sterile conditions, during which the nerves were cut into 0.5-1 cm segments and the nerve fascicles dissected from the nerve segments. Fascicles were then placed for one week in HuSC growth medium (DMEM supplemented with 10% FBS, 2 μM forskolin, 10 nM heregulin and 50 μg/ml gentamicin). Next, the fascicles were dissociated into a single cell suspension (Passage 0, P0 cells) and plated onto laminin-coated flasks (1 μg/cm²; Sigma, St Louis, Mo.). HuSCs were cultured to 60-80% confluence then trypsinized and split (P1). HuSCs were further passaged to P2 before use for transplantation. The purity of HuSCs was confirmed using immunocytochemistry for S100 (1:200, DAKO, Catalog#: 2031129-2, Carpinteria, Calif.) a protein highly expressed in SCs (Chelyshev and Saitkulov, Usp. Fiziol. Nauk., 31:54-69 (2000)). In all cases the purity of HuSCs was higher than 80%. HuSCs were used for transplantation at P2, either from ongoing cultures (fresh) or following their thawing and expansion to confluency from cryopreserved stocks. For the cryopreservation cohort, HuSCs were cryopreserved at P2, then later thawed and cultured for 24-48 hours before use. For comparing the growth curves of HuSCs that had been derived from different donors, some cells were maintained to P4. At each harvest, total cell counts were obtained by staining HuSCs for Syto24 (1:4, Invitrogen, Catalog#57020, Carlsbad, Calif.) and for dead cells, Sytox Green (1:4, Invitrogen, Catalog# S7559).

Contusive Spinal Cord Injury:

To enact a contusive SCI to the thoracic spinal cord, the MASCIS NYU impactor (Gruner, J. Neurotrauma, 9:123-8 (1992)), a well characterized and reproducible model, was employed. For these studies, a mild severity SCI was used to examine HuSC transplantation based upon preliminary studies that showed significantly larger lesions in immunocompromised animals compared to normal rats. Prior to surgery, animals were anesthetized with 4% isoflurane and 1.5 liters/min of oxygen in a designated induction chamber. Next, ketamine (70 mg/kg) and xylazine (5 mg/kg) were injected intraperitoneally for long-lasting anesthesia during surgical procedures. Under sterile conditions, a longitudinal incision was made along the back of the animal and a laminectomy was performed to expose the dorsal spinal cord at the thoracic (T8) vertebral level without disrupting the dura mater underneath. The exposed spinal cord was injured by dropping a 10 gr. rod from a height of 6.25 mm. To ensure consistency in the injury severity, the parameters of the impact were monitored and animals excluded when the impact parameters were determined to be out of range: height or velocity errors >7% from the expected value or if the compression distance was not between 0.75 to 1.25 mm (Patel et al., J. Neurotrauma, 27:789-801 (2010); Pearse et al., J. Neurotrauma, 21:1223-39 (2004a)). After SCI, the overlying musculature was sutured and the skin closed using wound clips.

HuSC Transplantation:

Prior to spinal cord implantation, HuSCs at P2 underwent three washes with medium to remove mitogens, laminin, and bovine products and then were trypsinized, collected for centrifugation, and then re-suspended in DMEM-F12 for counting. HuSCs were prepared in 10 μl aliquots at a density of 50,000 cell/μl in DMEM-F12 medium and kept on ice for a maximum of two hours prior to implantation. Cell injection occurred at four weeks after SCI, modelling the clinical protocol. Animals were anesthetized with ketamine/xylazine and the injured spinal cord exposed by removing the overlaying scar tissue while avoiding unintended injury to the underlying dura mater and spinal cord. Following spinal cord re-exposure animals were fixed by the spinal process rostral to the injury site (T7) with a spinal clamp attached to a stereotactic device (Narishige instruments, Catalog#SR-5R/STS-B, Tokyo, Japan). Upon fixation of the spinal process, a 10 μl Hamilton syringe was loaded with 8 μl of HuSCs in suspension. The syringe was attached prior to a liquid silicon pre-coated, pulled-and-beveled glass capillary needle (˜120 μm diameter). Once fixed in a stereotactic micromanipulator, the needle was lowered through the dura mater at the center of the lesion, which was visualized by a darkened discoloration of the spinal tissue. After lowering the needle to a depth of 1.0 mm into the spinal cord, an injection of 6 μl of HuSCs (300,000 cells total) was performed at a rate of 2 μl/min using an automatic microinjector (Quintessential Microinjector, Stoelting, Wood Dale, Ill.). The needle was kept in place for an additional 3 minutes to prevent leakage upon withdrawal. A concentration of 50,000 cells per μl was chosen for HuSC dose based upon the 2-3 times larger size of the cells compared to that of rat SCs. Following HuSC implantation, animals received the same postoperative care described after SCI. HuSCs from at least two different donors were prepared and used for transplantation, independently in different hosts, on any given day. To improve the long term survival of HuSCs in this xenotransplant paradigm, animals received additional immunosuppression with anti-Asialo GM1 antibody (50 μl intraperitoneal; Wako, Catalog#986-10001, Richmond, Va.) every 3 days starting at 3 days before transplantation to block natural killer (NK) cell activity (Drewinko et al., Invasion Metastasis, 6:69-82 (1986)).

### Results

Purity, Viability and Growth Curves of Donor HuSCs:

HuSCs were obtained in high numbers from the six cadaveric and organ donors (male and female, aged 20 to 62). Cultured HuSCs presented a classic spindle shaped cell morphology, S100 immunoreactivity and developed a swirling pattern of cell orientation when grown on laminin-coated flasks. At the time of transplantation, HuSC viability was higher than 98% for all donors, while purity (measured as the percentage of cells immunoreactive for S100) was greater than 90% for all donors except one, which exhibited a purity of 83%. After sural nerve harvest, similar initial numbers of HuSCs were obtained from all six donors and the HuSC growth curves from each donor showed exponential expansion of cells, with growth rates highest in younger donors. The average time for the HuSCs to reach P4 confluency was 42±2 days.

The Persistence of HuSCs after Transplantation:

Though stereological assessment of HuSC numbers in the 3 day cohort was not performed, NuMA or p75NTR and GFAP or Hoechst stained sections from the injured, transplanted spinal cord segment showed a dense implant within the lesion at this time. Hematoxylin, eosin- and luxol fast blue-stained sections revealed that the lesion site was largely filled by the HuSC implant with little cavitation.

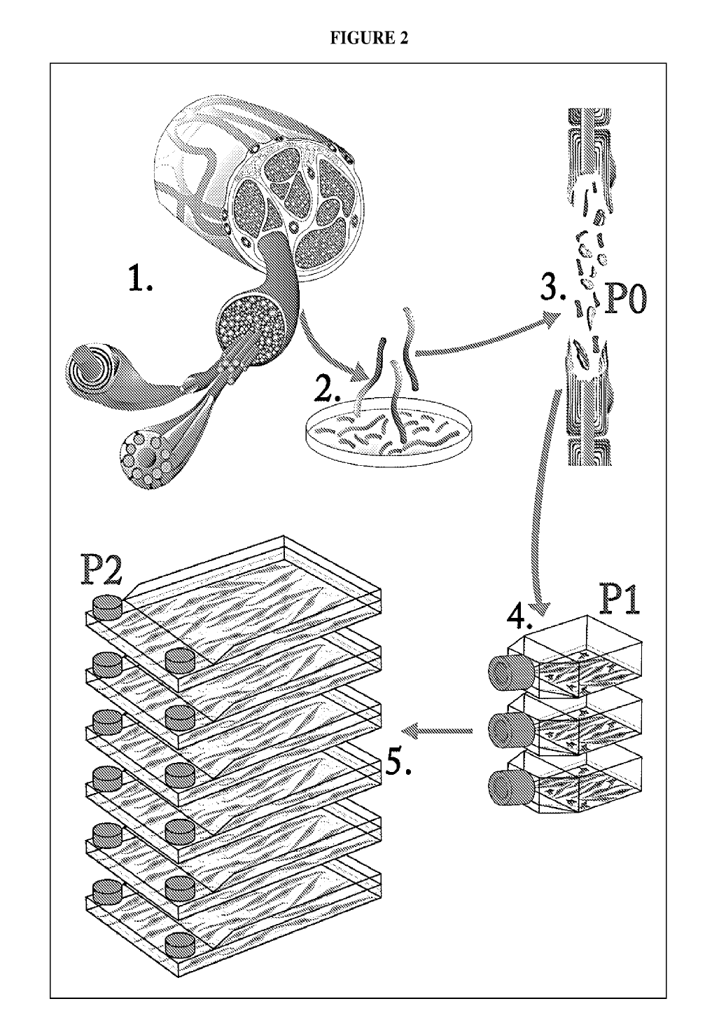

The quantitative evaluation of NuMA+HuSC profiles was performed in 29/30 rats from the 6 weeks cohort (1 animal was euthanized pre-endpoint due to a severe skin infection that did not respond to treatment). These animals received HuSCs originating from one of the 6 donors. The HuSCs prepared from donors D1, D2, D3 and D4 were grown to P2, cryopreserved and then thawed for transplantation, while for donors D5 and D6, the HuSCs were harvested, grown to P2 and used for transplantation without cryopreservation (fresh). The presence of NuMA+HuSC profiles within the injured spinal cord segment was confirmed in 25 out of 29 immunostained preparations. When expressed against the original number of transplanted HuSCs, the percent cell survival post-implantation for the 6 week cohort ranged from 0 to 23.9%, with the average survival rate across all donors and animals being 3.9±1.1%. Stained sections from the injured, transplanted spinal cord segment for NuMA and GFAP from an animal with a high HuSC survival rate at 6 weeks shows a compact implant, with scattered NuMA+ nuclei in a pocket of lesioned tissue that is surrounded by several small cysts (FIG. 2D, 2K). The survival rate of HuSCs within the injured spinal cord was significantly higher in those animals receiving cryopreserved cells (5.9±1.1%, n=19) as opposed to those implanted with freshly prepared HuSCs (0.8±0.1%, n=10; p<0.001). The number of days that HuSCs were maintained in cryopreservation before their preparation for transplantation presented a positive and significant correlation with the HuSC survival rate (p<0.01, R2=0.29). The donor with the longest period of HuSC cryopreservation prior to use (D1, 224 days) exhibited the highest average survival rate for transplanted HuSCs, 9.85±4.6%.

The presence of NuMA+HuSCs cell profiles was examined in 11/23 animals from the 6 months post-implantation cohort (12 animals were either euthanized or found dead pre-endpoint, Table 3). These animals received HuSCs originating from one of the four cryopreserved-only donors (D1 and D2, 4 animals; D3, 2 animals; D4, 1 animal). In comparison to 6 weeks post-implantation, fewer NuMA+HuSCs were detected within the injured spinal cord at 6 months. An average survival rate for HuSCs of 1.8±0.6% was obtained for the animals that reached endpoint, with HuSCs absent in 1 sample. NuMA and GFAP stained tissue sections of the injured, implanted spinal cord segment from an animal with the highest HuSC survival rate at 6 months shows a few scattered NuMA+ nuclei in groupings that were interspersed within the lesion and perilesional areas. The number of days that HuSCs were cryopreserved before their preparation for transplantation also presented a positive and significant correlation with the HuSC survival rate at 6 months post-implantation (p<0.05, R2=0.53). The donor with the longest period of HuSC cryopreservation prior to use (D1, 228 days) presented an average survival rate of 3.76±1% for transplanted HuSCs among the animals that reached the 6 month endpoint.

Low Proliferative Rate of HuSCs Following Transplantation with No Evidence of Tumorgenicity:

Assessment of NuMA+HuSC proliferation was obtained by quantification of their co-immunoreactivity with human-specific Ki-67. Examination of the implant after double staining revealed very few transplanted HuSCs that were actively undergoing proliferation at 6 weeks (0.09±0.05%) or 6 months (0.35±0.25%) after transplantation. Those isolated HuSCs co-labelled for Ki-67 and NuMA+ were found primarily at the boundary of the implant. An absence of human-specific Ki-67+ cell profiles was observed in 23/29 and 7/11 of the animals from the 6 week and 6 month cohorts, respectively. In contrast, significant Ki-67/NuMA co-immunoreactivity was obtained in the positive control sample, a section of tissue from a human Schwannoma biopsy. No significant differences in Ki-67+ cell counts were observed in transplanted animals from different donors or across times post-transplantation or was there a correlation between the degree of HuSC persistence and the recorded proliferation rates.

The low proliferative index of HuSCs post-implantation was confirmed by blinded, neuropathological examination of hematoxylin, eosin- and luxol fast blue-stained tissue sections, where the presence of solid tumors or active mitosis was not observed within the spinal cord along the entirety of the neural axis. In addition, no unexpected pathological findings were found along the neural axis in animals analyzed from all post-transplantation time points. In contrast, after spinal implantation of the rat G6 glioma cell line, a positive control for tumorgenicity, tissue sections presented an extremely high cellular density, indicative of active mitosis, with putative neoplasm formation.

Limited HuSC Biodistribution Along the Neural Axis:

The distribution of NuMA+HuSC profiles within the spinal cord was examined relative to their initial site of deposition within the lesion epicenter. Whereas the migration of SCs across adjacent segments of damaged nerve is critical to their reparative actions following peripheral nerve injury, it has been previously demonstrated that the distribution of rat SCs is highly restricted to lesion implant sites when they are injected into the injury epicenter after contusive SCI (Pearse et al., Glia 55:976-1000 (2007); Wang and Xu, Exp. Neurol., 261:308-19 (2014)). The biodistribution of NuMA+HuSCs was similar to that of rat SCs with cells being restricted to the injury cyst and immediate lesion penumbra across all time points following intraspinal injection into the injury epicenter. The biodistribution of HuSCs was confirmed with a human-specific P75NTR antibody, which also displayed a similar restricted localization of immunoreacive profiles within the lesion.

Temporal evaluation of the biodistribution of NuMA+HuSCs revealed that at 3 days post-transplantation immunoreactive cells were localized to the lesion and injury penumbra, with a few scattered cells present along the meninges near the entry site of the injection. HuSCs were neither found within the dorsal roots or ganglia nor outside the injured (2 cm) spinal cord segment. NuMA+HuSC implants existed as either a single, densely packed deposition of cells (8/16) or as multiple foci of cell collections (8/16). At 6 weeks and 6 months, NuMA+HuSCs were not found outside the lesion site and were fewer in number and more dispersed within the lesion. HuSC implants at 6 weeks showed cell groupings within the lesion (15/25) or were scattered without implant foci (10/22). At 6 months post-transplantation, only a few scattered NuMA+HuSCs were identified within the lesion. No donor-dependent differences in NuMA+HuSC biodistribution were found.

Host Glial Cell Response to HuSC Implants:

Co-staining with the astrocyte marker, GFAP, and NuMA or human P75NTR for HuSCs, revealed a large number of astrocytic processes as well as cell bodies intermingling with HuSCs within the lesion at 6 weeks and 6 months post-implantation, but not at 3 days. This interaction is in contrast to syngeneic rat SC implants where implanted SCs and host astrocytes usually form distinct interfaces with very limited interaction between them (Flora et al., Cell Transplant., 22:2203-17 (2013); Ghosh et al., Glia, 60:979-92 (2012); Patel et al., J Neurotrauma, 27:789-801 (2010); Pearse et al., Glia 55:976-1000 (2007)). Subsequent quantification of the amount of GFAP immunoreactivity within the rostral and caudal host spinal cord, immediately adjacent to the HuSC implant, revealed a temporal reduction in the average density of astroglial reactivity from 6 weeks (39.9±3 and 38.2±2.5% of total pixels for rostral and caudal segments, respectively) to 6 months (25±2.6 and 23.4±2.8% of the total pixels for rostral and caudal, respectively; p<0.001) post-transplantation. There was no correlation between the number of NuMA+HuSCs present and the density of GFAP immunoreactivity rostral or caudal to the implant at either post-transplantation time.

Next, the cellular immune reaction to the implanted HuSCs after SCI was measured by performing estimated counts of ED1+ cells within the rostral and caudal halves of the injured-implanted spinal cord segment. At 6 weeks after HuSC transplantation, an estimated average of 1,890±260 and 1,920±380 ED1+ cells per section was recorded for the rostral and caudal 500 μm segments, respectively. At 6 months following HuSC implantation, less ED1 immunoreactivity was observed, with estimated counts of 270±50 and 330±50 ED1+ cells in the rostral and caudal regions, respectively. Though there was a significant, temporal reduction in the cellular immune response post-implantation, no correlation existed between the number of NuMA+HuSCs within the injured spinal cord and the estimated ED1+ cell counts at either time point.

Axon and Endogenous SC Responses to HuSC Transplantation:

At 3 days after injection into the injured spinal cord, HuSC implants exhibited a dense cellular immunoreactivity for human-specific P75NTR within the lesion-implant site in an analogous deposition to that of NuMA. Stained cells exhibited the characteristic spindle-shaped morphology of SCs. Co-staining with NF-L showed numerous host axons within the penumbra of the lesion, surrounding P75NTR+HuSCs, though at this time there was no penetration of these axons into the lesion-implant proper. Immunoreactivity for either multi-species or human-specific P0 was not observed within the injured spinal cord at 3 days after HuSC transplantation.

At 6 weeks after HuSC transplantation, cellular immunoreactivity for human P75NTR was found more dispersed through the lesion in a patterning that mirrored NuMA, and numerous NF-L+ axons were found to have penetrated into the implant, intermingling with human P75NTR+HuSCs. A parallel alignment of human P75NTR+HuSCs with NF-L+ axons was observed. Staining for multispecies P0 at 6 weeks showed myelin profiles at the lesion-implant interface, but far fewer immunoreactive profiles for myelin within the lesion-implant. Density measurements of multi-species P0 immunoreactivity within the lesion-implant, as measured by the total number of immunoreactive pixels, provided an average measurement of 1.68±2.4×105 pixels. To evaluate HuSC myelination, tissue sections stained with the human specific P0 antibody and NF-L were evaluated. Whereas positive control human nerve samples showed human-specific P0 staining, immunoreactivity for human-specific P0 was negative in all HuSC implanted spinal cord samples. Confirmatory experiments employing cryosectioned tissue sections rather than those paraffin embedded for two animals with a 6 week survival period post-transplantation also showed an absence of human-specific P0 staining within the injured spinal cord segment. Density measurements of NF-L, expressed as immunoreactive pixels, provided an average measurement of 1.79±2.7×105 pixels with the HuSC implant at 6 weeks post-transplantation. Correlation analysis of NF-L and multispecies P0 with the number of NuMA+HuSC within the lesion-implant at 6 weeks post-transplantation showed a significant, positive correlation for both NF-L (FIG. 71; R2=0.21, p<0.05) and multi-species P0 (FIG. 6S; R2=0.40, p<0.001). While there is a correlation between NF-L+ axons and P0+ profiles with NuMA counts, the numbers of myelinated axons within the HuSC implant are low at 6 weeks.

At 6 months after HuSC transplantation, scattered human P75NTR+ cell profiles were identified throughout the lesion that approximated the localization of NuMA+. In contrast to 6 weeks post-transplantation, the lesion-implant site contained a large number of NF-L+ axons and P0+ myelin rings in all animals (11/11). The considerable disparity in the number of NuMA+/human P75NTR+HuSC profiles and the extent of immunoreactivity for multi-species P0 implied that large numbers of host rat SCs had breached the lesion to myelinate the NF-L+ axons present within. Density measurements of P0 immunoreactivity within the lesion-implant, as measured by the total number of immunoreactive pixels, presented average measurements of 1.8±0.4×10⁶ pixels. However, like the 6 weeks cohort, immunoreactivity for human-specific P0 was negative in all samples from the 6 month post-transplantation group. Density measurements of NF-L, expressed as immunoreactive pixels, provided an average measurement of 5.8±1.2×10⁶ pixels. The immunoreactive density values for NF-L and multispecies P0 within the lesion-implant at 6 months after HuSCs transplantation were ˜10-fold greater than those at 6 weeks. However, similar to 6 weeks, there remained a significant, positive correlation between the density of NF-L (R2=0.57, p<0.01) or multispecies P0 (R2=0.57, p<0.01) immunoreactivity and the number of NuMA+HuSCs present within the lesion at 6 months post-transplantation.

The degree of axonal retraction rostral from the lesion-implant site was quantified using measurements of NF-200 density in a 500 μm spinal cord segment encompassing the lesion-implant interface. The area of NF-200 immunoreactivity (in pixels) was expressed as a percent of total pixels measure within the 500 μm spinal cord segment. An increase in the amount of axon retraction was observed, with an average of 30.6±2.4% and 44.4±3.2% pixel coverage at 6 weeks and 6 months post-transplantation, respectively (p<0.05). Linear correlation analysis revealed that no correlation existed between the density of retracted NF-200+ axons in the rostral spinal cord and the number of NuMA+HuSCs present within the lesion at 6 weeks and 6 months post-transplantation.

Higher NuMA+HuSC Counts Showed a Positive Correlation with the Degree of White Matter Preservation:

To evaluate the effects of HuSC transplantation on secondary tissue pathology following SCI, including cyst expansion and host tissue damage, stereological quantification of preserved white and grey matter volumes was performed. A one centimeter length of the spinal cord encompassing the injury-implant at its center was analyzed from animals in the 6 week (27/29) and 6 month (11/11) cohorts. Two samples from the 6 week group were excluded due to tissue sectioning issues.

The average volumes of preserved white matter (6 weeks, 1.05±0.02 mm³; 6 months, 1.15±0.02 mm³) and healthy appearing grey matter (6 weeks, 0.59±0.02 mm³; 6 months, 0.73±0.05) were measured following HuSC transplantation. Linear correlation analysis showed a positive correlation between the number of NuMA+HuSCs present within the injured spinal cord and the volume of preserved white matter at both 6 weeks (R2=0.4758, p<0.0001) and 6 months (R2=0.41, p<0.05) post-implantation. Micrographs show spinal cord sections from an animal with a low HuSC survival rate and an animal with a high HuSC survival rate at 6 weeks post-transplantation. Similarly, at 6 months after grafting, HuSC survival showed a positive correlation with white matter preservation; in animals with fewer HuSCs, less white matter preservation and more numerous cysts were observed.

### Discussion

To address the question whether HuSCs were safe when transplanted after SCI, these studies sought to emulate the clinical use of HuSCs by employing unlabeled (virus-free) cells that were generated using the same protocol used clinically and the comparative examination of HuSCs from multiple donors. Here, it is demonstrated that HuSCs persisted in the contused nude rat spinal cord for up to 6 months after transplantation and, analogous to rodent SCs, they presented limited migration, a low proliferation rate and no tumorgenicity potential. Although no donor differences were seen in temporal HuSC persistence after spinal cord transplantation, significantly greater HuSC survival was obtained when the cells were used from cryopreserved stocks rather than employed as fresh isolates. Numbers of surviving HuSCs within the lesion site positively correlated with the extent of preserved white matter of the injured cord segment and no adverse effects of the transplants on tissue pathology were observed at 6 weeks and 6 months. Furthermore, HuSCs were supportive of significant NF+ axon growth into the lesion-implant and were able to intermingle with host astrocytes, both at the implant interfaces and within the lesion-implant proper. The transplantation of HuSCs into the injured rat spinal cord also led to significant host SC migration into the lesion-implant areas, which then led to extensive myelination of axons that had entered the lesion-implant. No significant differences in SC morphology, purity or viability were observed across donors, however, those of younger age exhibited a higher SC proliferation rate.

In an unexpected finding, the use of HuSCs as freshly prepared cell isolates rather than from cryopreserved stocks led to lower (but still acceptable) survival rates following transplantation. The percent HuSC survival rate exhibited a positive correlation with the length in days that HuSCs were maintained in cryopreservation. A possible explanation for this finding is that the cryopreservation protocol, which produces some degree of cell loss, may act as a selection method.

The data demonstrated that HuSCs persist within the contused spinal cord for up to 6 months after transplantation in almost all animals, though the survival rates of HuSCs in these experiments were lower and more variable than those of previously published reports employing syngeneic rat SC transplantation after contusive SCI. At 6 weeks after transplantation, the survival of rate of HuSCs was significantly lower (3.9% versus 15-20%) than that reported for syngeneic rat SCs. Furthermore, the numbers of HuSCs decreased further from 6 weeks to 6 months after transplantation, which contrasts with that of rat SCs where the number of surviving cells post-transplantation show little change from 6 weeks to 3 (Barakat et al., Cell Transplant, 14:225-40, (2005)) or 6 months (Wang and Xu, Exp. Neurol., 261:308-19 (2014)). In addition to quantifying HuSC survival rates, proliferation indices using Ki-67 also were measured. Similar to previous work with rat SCs, where proliferation rates have been measured in the <5% range (Patel et al., J Neurotrauma, 27:789-801, (2010); Wang and Xu, Exp. Neurol., 261:308-19 (2014)), HuSCs exhibited very low amounts of cell proliferation temporally after transplantation. Furthermore, upon neuropathology examination, no abnormal cell mitosis or presence of solid tumors was observed within the lesion-implant as well as along the entirety of the spinal cord, demonstrating the very low tumorgenicity potential of HuSCs.

The data demonstrate that the numbers of HuSCs found within the lesion-implant site positively correlated with the amount of host preserved white matter, axon growth (NF density) and endogenous SC myelination at both 6 weeks and 6 months after transplantation, responses similar to those reported previously for rat SCs (Bamber et al., Eur J Neurosci 13:257-68 (2001); Guest et al., Exp. Neurol., 148:502-22 (1997b); Xu et al., J. Neurocytol., 26:1-16 (1997); Xu et al., Exp. Neurol., 134:261-72 (1995a); Xu et al., Eur. J. Neurosci., 11:1723-40 (1999)). While density measures of fluorescent-conjugated antibody immunoreactivity is not a decisive method for quantifying protein presence, as the amount of staining may not correlate with the amount of antigen in the tissue and it exhibits a significant non-linear behavior (Taylor and Levenson, Histopathology, 49:411-24 (2006)), substantial structural changes within the implant-lesion comprising an increased number of stained profiles indicated that marked alterations in cell and axon profiles occurred in the lesion-transplant with time. Strong P0 immunoreactivity observed specifically at 6 months after transplantation showed that HuSCs are able to attract endogenous SCs in a similar fashion to that found in rat SC experiments (Hill et al., Glia, 53:338-43 (2006); Pearse et al., Glia, 55:976-1000 (2007)). Although it has been reported that HuSCs are able to myelinate axons when transplanted into a demyelinated spinal cord lesion (Brierley et al., Cell Transplant, 10:305-15 (2001)) or a peripheral nerve (Levi and Bunge, Exp Neurol 130:41-52 (1994); Levi et al., J. Neurosci., 14:1309-19 1994), it could not be confirmed in the injured spinal cord with multi-species or human-specific P0 antibody that HuSCs could myelinate axons in our experiments. Differences in the environment, whether the x-irradiated spinal cord or peripheral nerve, or the preparation of HuSCs, which may have produced selection of a cell population, in these studies may explain this discrepancy. It is also possible that our investigation at 6 weeks may not have allowed sufficient time for myelination. Robust rat SC myelination in a contusion SCI paradigm has been reported at 3 months after transplantation (Kanno et al., J. Neurosci., 34:1838-55, (2014); Pearse et al., Glia, 55:976-1000 (2007); Wang and Xu, Exp. Neurol., 261:308-19 (2014)). On the other hand, at 6 months after transplantation, only a few scattered HuSCs were detected in the samples, which potentially limited the ability of detecting myelinating cells. Examination of intermediate time points or the use of larger numbers of HuSCs may be needed to better answer the question as to whether culture expanded HuSCs can myelinate axons within the lesion-implant following SCI and transplantation in a xenotransplant paradigm.

To summarize, the data demonstrated that HuSCs can persist long-term after transplantation (up to 6 months) and have a favorable toxicity profile that is exhibited by a limited biodistribution, a low proliferation rate and an absence of tumor formation or abnormal pathological changes. HuSCs, in combination with a robust endogenous host SC response, lead to white matter protection, axon growth support, and myelination within the injured cord segment. These studies provide evidence that HuSCs do not display histological changes indicative of toxicity while presenting signs of positive influence of transplanted HuSCs on host tissue pathology that are similar to those found in previous work that employed rodent SCs in an analogous SCI paradigm.

### Example 3

A Phase 1 clinical trial was conducted to evaluate the safety and feasibility of autologous human SCs (ahSC) transplantation into the injury epicenter of six subjects with subacute SCI (spinal cord injury). The trial was an open-label, unblinded, non-randomized, non-placebo controlled study with a dose escalation design. The primary end point was to evaluate the safety through a 1 year follow-up when ahSCs were administered at one of three doses within 72 days of injury to participants with complete thoracic SCI.

### Methods

Sural Nerve Harvest:

In the operating room, the medial calf above the medial malleolus was infiltrated with local anesthetic with 1% epinephrine. The sural nerve was identified and an approximately 15 cm segment was harvested for ahSC preparation by sharp dissection.

Cell Processing:

The sural nerve was dissected, and fascicles were pulled from the epineurium and transferred to a triangular T-75 flask (Corning, Oneonta, N.Y., USA) placed in an incubator at 37° C. with 8% CO₂. Culture medium, which contained 1× Dulbecco's modified Eagle medium (Life Technologies, Grand Island, N.Y., USA), 10% fetal bovine serum (Hyclone, GE Healthcare Life Sciences South Logan, Utah, USA), 2 mM forskolin (Sigma-Aldrich, St. Louis, Mo., USA), 10 nM human recombinant heregulin 01 (Genentech, South San Francisco, Calif., USA), 4 mM L-glutamine (Life Technologies), 0.064 mg/ml gentamicin (APP Pharmaceutical/Fresenius Kabi USA, Lake Zurich, Ill., USA), was changed every other day. On day 7±2 the dissociation enzyme solution that contained neutral protease NB (2 DMCU/ml; SERVA Electrophoresis GmbH, Heidelberg, Germany), collagenase NB1 (0.5 PZU/ml; SERVA Electrophoresis GmbH) in 1× high-glucose Dulbecco's modified Eagle medium (Life Technologies) supplemented with 3.1 mM CaCl₂ (International Medication Systems Limited, South El Monte, Calif., USA), was added to the fascicles and placed inside the incubator overnight. The fascicles were dissociated, D-10 (Life Technologies) (10 ml) was added to a tube containing the fascicles, which was centrifuged at 150×g for 5 min at 4° C. to pellet the cells. The cells were washed and plated onto mouse laminin-coated (1 μl/cm2, with a stock concentration of 1 mg/ml; Sigma-Aldrich) plates using the culture medium, then fed with culture medium every 3 days. After 7 days, cells generally reached 80-90% confluence. The viable cell counts of final harvests before the transplantation ranged between 11.4-436.8 million cells for all six products. During the trial, the cell processing techniques were optimized to yield higher cell recovery rates, hence the wide range of viable cells. These modifications were tested and validated before being implemented into the trial; they led to the routine yield of over 200 million SCs. The SCs purity by immune staining ranged from 92.2-98.7% and the SCs viability also ranged from 93.2-97.2%. The final cell products were washed to remove mitogens, laminin, and bovine products. Several controls were employed throughout the manufacturing process to ensure that the product was essentially free of process-related contaminants, including the wash steps described above and release testing of the final product. Mycoplasma (via PCR) testing was negative for all six ahSC products infused; final endotoxin levels were less than 0.2 EU/kg; Gram stains were negative. Post-transplantation sterility results after 14 days of culture were also negative for aerobic, anaerobic, and fungal organisms.

For immunostaining, SCs were plated onto laminin-coated four-well glass chamber slides at 50,000 viable cells per well and fixed the following day with 4% paraformaldehyde (Electron Microscopy Sciences, Hatfield, Pa., USA). For identification of SCs, an anti-human S100 (final dilution 1:200; Dako, Dallas, Tex., USA) primary antibody was used. Within the peripheral nerve tissue, SCs are the only cell type that expresses S100 protein. To identify the second major cell type that is a constituent in the nerve dissociate, an antibody was used that recognizes fibronectin, which is abundantly present in fibroblast extra-cellular matrix; an anti-fibronectin (final dilution 1:200; Santa Cruz Biotechnology, Santa Cruz, Calif., USA) primary antibody was used. The secondary antibodies used were a solution combination of Alexa 488 goat anti-rabbit IgG and Alexa 594 goat anti-mouse IgG (Life Technologies) diluted (1:200) in 1.5% normal goat serum and 1×DPBS (Life Technologies). All wells received Hoechst nuclear marker (final dilution 1:1,000; Life Technologies). Fluorescent images were obtained using a fluorescent microscope.

Dose escalation was performed in three cohorts with the first cohort receiving 5 million cells in 500, the second cohort receiving 10 million cells in 100 μl, and the third cohort receiving 15 million cells in 1500. The dose to be administered plus back-up aliquots of cells were placed on ice to transport to the operating room for transplantation.

Intramedullary Injection of ahSCs:

After induction of general anesthesia and intubation, the subjects were placed in the prone position on a Jackson Table (Mizuhosi OSI, Union City, Calif.) (http://www.mizuhosi.com/jacksonSpine.cfm). Pre-incisional intravenous antibiotics were administered (Vancomycin 1 g/Ceftriaxone 2 g). Somatosensory and motor evoked potentials were used for spinal cord and nerve root monitoring. Based on the pre-operative imaging including MRI and CT scan, the surgical level was determined and a lateral spine x-ray was used intra-operatively to localize the site of prior surgery. All patients had previously undergone decompressive laminectomy and/or spinal re-alignment and instrumented fusion after injury. A priori, surgical planning for stabilizing instrumentation was performed to avoid placing pedicle screws and cross link at site of injury epicenter to avoid instrumentation artifact. The middle portion of the prior midline skin incision was reopened to expose the surgical transplantation site. Sharp dissection of very adherent scar tissue down to the dura was needed in all patients. Additional partial laminectomy above or below the injury site was needed in a few patients to obtain appropriate dural exposure at the transplantation site. The injections were performed with maintenance of the posterior spinal instrumentation in place.

Intra-operative ultrasound imaging (Hitachi HI Vision Ascendus, Hitachi Medical Systems Europe Holding AG, Switzerland/12 MHz linear array transducer on an IU22 scanner (Hitachi Aloka Medical America, Inc, Wallingford, Conn.)) was used to visualize intramedullary changes, especially cystic cavities. This information was used in addition to estimation of the borders of the traumatic lesion based on the extent of the altered intramedullary signal changes on T1 and/or T2 images which are typical of SCI. According to the ultrasound and MRI findings, the extent of dural opening was determined. Dural tack up sutures were placed. Using the magnification and illumination of the operating microscopic, the dorsal surface anatomy was exposed. Extensive untethering or fenestration of a spinal cord cyst was avoided. The table mounted syringe positioning device designed by Geron Corp. was anchored to the operating table in a sterile fashion and its components assembled.

Special care was taken to avoid damaging superficial blood vessels. The ahSC dose was predetermined according to the escalation parameters. Gentle swirling and tapping of the vial was required to disperse the cells prior to syringe loading. A commercially available 250 μl Hamilton syringe with 10 μl marks was used to draw up and deliver the cells through a 26S-gauge needle tip. The syringe was mounted into the syringe positioning device and lowered into proximity of the target injection site. Apnea was invoked to reduce spinal cord excursions due to ventilation, with continuous infusion of 100% oxygen through the endotracheal tube to maintain oxygen saturation of the blood. Using the syringe positioning device microstage and depth adjustment, the injection needle was advanced to the pial surface and a small pial opening created with a tip of a #11 blade to prevent cord compression during needle insertion. The needle was then carefully inserted perpendicular to the pial surface until the tip reached the desired depth calculated based on the pre-operative MRI and intra-operative ultrasound. The site targeted for injection was the injury epicenter—most commonly 3-4 mm below the pial surface. The cells were infused over approximately four minutes, followed by an additional one-minute dwell time to avoid reflux of cells along the needle tract. Upon withdrawal of the needle, special attention was given to the pial surface to document cell reflux from the needle tract. After the intramedullary injections, the dura was closed in a watertight fashion with 5-0 Prolene sutures, ultrasound was repeated, and the dura covered with Duragen Plus (Integra, Plainsboro, N.J.).

Primary Endpoints for Safety:

Primary outcome measures for safety were protocol compliance, feasibility, AEs, and stability of neurologic level. Compliance and feasibility were chosen due to the complexity of the protocol, particularly related to the need for two surgical procedures in the subacute time period in addition to the primary decompression and fixation surgery. Evaluation of AEs is a key indicator of safety. Stability of the neurologic level was chosen because of the plan to inject cells into the epicenter of the subacute lesion and the possibility of interference with the evolving spinal cord cavitation or resolving inflammatory process that might lead to neurologic deterioration. Deviations from the protocol were documented to monitor compliance and feasibility. All AEs and SAEs were recorded and a determination of relatedness to ahSCs was made. To document neurologic change the ISNCSCI was used along with the AIS grading of completeness.

Secondary Endpoints for Safety:

Secondary outcome measures for safety were absence of detectable mass lesion on MRI, the emergence of clinically significant neuropathic pain no greater than expected for a natural course cohort, and emergence of clinically significant muscle spasticity no greater than expected for a natural course cohort. Absence of detectable mass lesion was chosen because cell therapies in general carry the inherent risk of tumorigenesis, even though human SCs themselves carry a very low risk. Pain and spasticity were chosen as secondary safety endpoints because of the possibility that they could be negatively impacted by aberrant neuroplasticity. MRI scans of the thoracic spine without and with gadolinium were performed on a Siemens 1.5T magnet using metal artifact reduction sequences to limit instrumentation artifact. MR imaging was generally performed immediately post injury, 1-2 days prior to transplantation (baseline), day 1 and months 6 and 12 post-transplantation. Lesion volume was determined by using the free hand measurement tool on axial images to calculate the area of signal abnormality multiplied by the cut thickness. Pain was assessed using a combination of a pain drawing, the International SCI Basic Pain Dataset25, and the Neuropathic Pain Symptom Inventory. Spasticity was evaluated in the hamstrings and quadriceps using the Modified Ashworth Scale.

Exploratory Domains for Efficacy:

Though the primary endpoint for this trial was safety, multiple domains for preliminary indicators of efficacy were evaluated. Functional ability was measured with the Spinal Cord Independence Measure version III26 and the Functional Independence Measure®. To evaluate the burden of injuries sustained during the initial trauma the Injury Severity Score29 was calculated. Electrophysiology measures of somatosensory and motor evoked potentials were performed as a secondary evaluation of neurological connectivity, and sympathetic skin response as an evaluation of autonomic neurological connectivity. Bladder and bowel control was evaluated with the International SCI Lower Urinary Tract Basic Dataset30 and the International SCI Bowel Function Basic Dataset.31 The Patient Global Impression of Change was used to rate participant impression of change after enrollment.

Statistical Analysis:

As an open label study lacking a control group, statistical analysis is limited other than for the independent variable of cell dose. The primary endpoint analyses occurred at 12 months post-transplantation. All analyses were based on descriptive measures of the cohort and within-subject changes.

### Results

Participants:

Thirty-nine individuals were pre-screened. Nine individuals were enrolled and six received ahSC transplantation. Of the three that were enrolled but not transplanted, one self-withdrew a few hours after enrolling (and did not undergo a nerve harvest), one was withdrawn because the cells became contaminated prior to the scheduled transplantation, and one converted to AIS B between the nerve harvest and transplantation and was withdrawn prior to the scheduled transplantation.

ahSC Culture and Transplantation:

The mean (all data to follow presented as Mean±Standard Deviation) number of days between injury and nerve harvest was 14±11.7. The length of sural nerve harvested was 15.9±4.0 cm. The number of days between nerve harvest and transplantation surgery was 26±4.0. Transplantations occurred an average of 40±12.3 days post-injury. High SC purity at product release was obtained for all transplantations with the mean being 96.9±2.4%. After meticulous removal of the scar, intra-operative ultrasound was used to define the epicenter of the injury, the dura was opened, and the syringe positioning device was set up. Apnea was induced, mean duration 6 min 12 sec±30.0 sec, and the injection was performed over a mean duration of 4 min 10 sec±59 sec at a rate of 22.3±8 μl/min. In five of the six cases, ahSC efflux from the injection site after needle removal was of minimal volume, which was observed under the surgical microscope at high magnification and adsorbed onto cottonoid patties. In one subject, a larger volume of efflux was observed early during the injection and the injection was stopped for 30 seconds to adsorb the efflux, the needle was deepened by 1 mm, and injection resumed. No cells were delivered into the intrathecal space. Each syringe was pre-loaded with the dose to be delivered plus an extra 30 μl of cell suspension to enable adjustment for any efflux volumes.

In regard to induced apnea during cell injection, durations of up to 6 min 45 sec were well tolerated with suitable pre-oxygenation and initial CO₂ levels in the low-normal range. Careful timing of the onset and offset of apnea occurred between the surgical and anesthetic team, with each 30 second interval being stated out-loud. Post-apnea CO₂ values of 49-57 were not associated with cardiac instability or other adverse changes.

Primary Evaluation of Safety:

AEs were recorded from the time of enrollment through 12 months post-transplantation. The AEs were those reasonably expected for a trial enrolling participants shortly after a traumatic injury. Notably, there were no occurrences of deep vein thromboses, pulmonary emboli, meningitis, or pseudomeningoceles. One subject sustained significant poly-trauma with his injury and was not discharged from the acute hospital setting until just before 9 months post-transplantation. He also experienced the most AEs, twenty-one. Only one SAE occurred and it was determined to be not related to ahSC treatment. It was an overnight hospitalization to investigate a cardiovascular event 3 months post-transplantation. One subject experienced an altered sensation without numbness or pain in the T2 dermatome of both arms from the elbow to the axilla at 3 months post-transplantation. By 12 months the altered sensation had resolved in the left arm. This altered sensation was noted as an AE reviewed by the Medical Monitor and Data Safety Monitoring Board, and deemed not unexpected for SCI.

The ISNCSCI and AIS were used to evaluate neurologic changes. There were no motor changes in any of the participants. There were minor sensory changes in five participants that were similar to the natural changes that occur for these types of injuries during the first year of recovery. Specifically, one patient gained 2 sensory points from baseline, but lost 2 levels per the single neurologic level (SNL) due to the altered sensation described above; the zone of partial preservation (ZPP) descended 1 level. A second patient lost 3 sensory points, but there was no change in SNL or ZPP. A third patient gained 2 sensory points and the SNL descended 1 level within the ZPP. A fourth patient gained 2 sensory points, had no change in the SNL, but the ZPP descended 1 level. A fifth patient lost 3 sensory points and the SNL ascended 1 level, but the ZPP descended 1 level. The most change was observed in a sixth patient; at 6 months post-transplantation he converted from an AIS Grade A to B, which persisted at month 12. He gained 9 sensory points by month 12, but his SNL did not change.

Secondary Evaluation of Safety:

MR imaging at baseline demonstrated significant variability in intramedullary post-traumatic changes; lesion length varied from 2.5-7.6 cm with a mean of 5.6±2.1 cm and lesion volume varied from 0.8-4.1 cc with a mean of 2.3±1.2 cc. Whereas spinal cord transection was an exclusion, the degree of cord damage was severe and there were no limits set on lesion size. Lesion characteristics were at times obscured by the presence of instrumentation despite a priori surgical planning. In some cases it was difficult to distinguish structural injury versus peri-injury edema. The spinal cord was well decompressed at the time of the ahSC transplantation in all but one case in which there was an epidural fluid collection.

Participants were rescanned on Day 1 post-transplantation. Whereas small postoperative fluid collections were seen at the revision laminectomy site, there were no areas suggestive of intramedulluary hemorrhage resulting from the cellular injection, independent of the dose. On the 12 month MRI scan, there was atrophy of the spinal cord. In one case, there was atrophy and focal tethering of the spinal cord. Lesion length at 12 months varied from 2.2-6.9 cm with a mean of 4.8±1.9 cm. Lesion volume at 12 months varied from 0.8-1.7 cc with a mean of 1.0±0.5 cc. Lesion length and volume were significantly different between baseline and 12 months post-transplantation. For lesion length, using an unpaired two-sided T-test, the p value was 0.008 (Graphpad Instat 3.01). For lesion volume, using the same statistical comparison, the p value was 0.034.

Pain was monitored as a secondary outcome measure of safety. Three participants did not experience neuropathic pain either at pre-transplantation or at 12 months post-transplantation. Two participants experienced neuropathic pain at pre-transplantation, which had increased in severity at 12 months post-transplantation. One participant did not experience neuropathic pain at baseline but had developed neuropathic pain at 12 months post-transplantation. The neuropathic pains that were experienced by three individuals at 12 months post-transplantation were severe and interfered with various life activities. The presence of neuropathic pain in 50% of these trial participants at one-year post-SCI is consistent with longitudinal studies and not unexpected. This percentage is used only for comparison with published longitudinal data, but due to the small sample size the results should be interpreted with caution. Flexor and extensor spasticity in the lower extremities developed pre-transplantation to varying degrees in all participants and the changes 12 months post-transplantation were not unexpected.

Exploratory Evaluation of Efficacy:

Functional recovery as measured by the Functional Independence Measure® and the Spinal Cord Independence Measure III occurred as would be expected (Table 3, columns labeled as “FIM” and “SCIM III”), but was influenced by the severity of the associated trauma sustained during the initial injury. The Injury Severity Score ranges from 0-75 with twenty-five being considered “severe” and seventy-five being considered unsurvivable; it was used to evaluate the initial burden of trauma. The presence of a severe thoracic SCI automatically accounted for an Injury Severity Score of 16. One patient also sustained a rotator cuff injury and wrist fracture, which increased his Injury Severity Score to 20. A second patient sustained a significant brachial plexus injury along with scapula, clavicle, and rib fractures, which increased his Injury Severity Score to 24. A third patient sustained 3 fractures (femur, scapula, and thumb) and significant chest trauma (hemothorax, respiratory failure, pulmonary contusions), which generated an Injury Severity Score of 34. These three participants having a higher burden of injury is reflected by their lower functional ability scores at baseline.

A consistent reproducible somatosensory evoked potential waveform was not observed. Baseline electrophysiology was performed within 5 days prior to transplantation and again at 2, 6, and 12 months post-transplantation. The subject that converted to AIS B had detectable motor evoked potentials in both legs at both six and twelve months and three subjects were able to voluntarily activate electromyography signal in the legs when a Jendrassick maneuver was performed. All of this activity was below the level of clinical detection, i.e., in 0/5 scored muscles. The detection of sympathetic skin responses was complicated in subjects receiving anti-cholinergic medications because sweating was blocked pharmacologically via concomitant medication. Bladder and bowel control did not change. All participants rated their overall global impression of change as having improved by some degree at 12 months post-transplantation, though this could also be expected to occur in untreated individuals during the first year post-injury.

Rehabilitation in this trial was per standard medical care; activities were not prescribed by the trial. As such, standard medical rehabilitation is designed unique to the individual and injury. In general, however, inpatient rehabilitation consisted of a therapy program involving 3 hours each day, 5 days each week based on the individual's injury, rate of progress, and therapeutic milestones set by the clinical rehabilitation team. The six participants received an average of 6.6±2.1 (standard deviation) weeks of inpatient rehabilitation.

### Discussion

This study of six subjects transplanted with autologous purified SCs, evaluated for one year, showed no clear indication of adverse events specifically linked to the nerve harvest, cell transplantation procedure, or presence of the cells within the spinal cord. It is feasible to identify, appropriately obtain informed consent, perform a peripheral nerve harvest within 5-30 days of injury, and introduce an intra-spinal transplantation within 4-7 weeks of injury, even in individuals having sustained severe injury.

An initial time window of 5 days post-injury was set as a cut off for the nerve harvest and 42 days post-injury as a cut off for the ahSC transplantation in an attempt to maximize the potential neuroprotective properties of SCs while working within the required time for cell processing. The 5 day time window, however, proved to be difficult when trying to recruit participants within the context of the strict inclusion and exclusion criteria and in using a single center enrollment site. Approximately one year into the trial, approval was obtained to extend the cut off for nerve harvest to 7 days post-injury while maintaining the 42 day post-injury cut off for the transplantation. At the same time, the age cut off was extended to 60 years from 50 and the upper and lower limits of the liver functioning tests were removed from being exclusionary criteria. Extension of the age range to 60 years of age was determined to not impose any additional risk because a) individuals with significant health concerns would be excluded by the other criteria and b) negative effects of age (independent of health status) on co-morbidities after SCI are more frequent in elderly individuals, i.e., >60 years of age. Additionally, it is not uncommon for individuals having sustained an acute traumatic injury, like SCI, to have some lab values outside of the typically accepted ranges. The first two participants both had lab values for their liver function tests outside of the ranges initially listed in our criteria, yet the values were not ones that would raise safety concerns for the transplantation procedure. Approximately six months later the window was extended for the nerve harvest to 30 days post-injury and the transplantation to 72 days post-injury and added a second site for recruitment and screening procedures.

The changes in neurological function after transplantation were not clinically meaningful in 5 of 6 subjects, but this may reflect the severity of the enrolled thoracic spinal cord injuries, as demonstrated by the large lesion sizes, and the relative low number of transplanted SCs. Very few individuals with thoracic AIS A injuries will spontaneously convert to incomplete grades. One of the six subjects in this trial converted to AIS B. This represents approximately 16% of participants, which falls within the expected 18% rate of spontaneous conversion. It is also common to observe minor gains or losses in sensory levels as well as the single neurologic level in thoracic injury, most often limited to one segment. A deterioration of three or more thoracic sensory levels would be unusual and has been suggested as a marker to track safety in Phase I clinical trials targeting acute/subacute SCI. None of the six transplanted subjects experienced a loss of three levels. Additionally, none of the subjects experienced any neurologic or functional deterioration during the first days or weeks following the transplantation.

Persistent and severe pain is common after SCI, with approximately 60% experiencing neuropathic pain within one year post-SCI. Consistent with the natural course of neuropathic pain development after SCI, three to four out of our six participants, would be expected to experience neuropathic pain at one year post-injury. In the present study, three subjects experienced neuropathic pain at 12 months post-SCI. Thus, the ahSC implant was associated with neuropathic pain within expected frequency without any attenuation of the neuropathic evolution.

This entire document is intended to be related as a unified disclosure, and it should be understood that all combinations of features described herein are contemplated, even if the combination of features are not found together in the same sentence, or paragraph, or section of this document. The invention also includes, for instance, all embodiments of the invention narrower in scope in any way than the variations specifically mentioned above. With respect to aspects of the invention described as a genus, all individual species are considered separate aspects of the invention. With respect to aspects of the invention described or claimed with “a” or “an,” it should be understood that these terms mean “one or more” unless context unambiguously requires a more restricted meaning. If aspects of the invention are described as “comprising” a feature, embodiments also are contemplated “consisting of” or “consisting essentially of” the feature.

All publications, patents and patent applications cited in this specification are herein incorporated by reference as if each individual publication or patent application were specifically and individually indicated to be incorporated by reference.

What is claimed:

1. A method of producing a population of human Schwann cells, the method comprising
(a) incubating human fascicles with one or more mitogens for a priming period of three to fourteen days to produce primed fascicles,
(b) incubating the primed fascicles with one or more tissue dissociation enzymes to produce primed Schwann cells,
(c) culturing the primed Schwann cells at an initial P0 density of 10,000 cells/cm² to 15,000 cells/cm² for a period of time to achieve no greater than 90% confluence,
(d) expanding the population of Schwann cells by culturing the Schwann cells at an initial passage density of 6667 cells/cm² to 13333 cells/cm² for a period of time to achieve no greater than 90% confluence for at least two passages, and
(e) harvesting the population of human Schwann cells.

2. The method of claim 1, wherein the priming period comprises three to eight days.

3. The method of claim 2, wherein the priming period comprises eight days.

4. The method of any one of claims 1-3, wherein the mitogen is forskolin, heregulin, or a combination of forskolin and heregulin.

5. The method of any one of claims 1-4, wherein the human fascicles are extracted from human nerve tissue one day or more following dissection.

6. The method of claim 5, wherein the human fascicles are extracted from human nerve tissue about seven days following dissection.

7. The method of claim 5 or claim 6, wherein the human nerve tissue is sural nerve tissue.

8. The method of any one of claims 1-7, wherein step (b) comprises incubating the primed fascicles with the tissue dissociation enzyme for 12 hours to 24 hours.

9. The method of claim 8, wherein step (b) comprises incubating the primed fascicles with the tissue dissociation enzyme for 16 hours to 18 hours.

10. The method of any one of claims 1-9, wherein the tissue dissociation enzyme is a metalloprotease.

11. The method of any one of claims 1-10, wherein the tissue dissociation enzyme(s) is collagenase, neutral protease, or a combination of collagenase and neutral protease.

12. The method of any one of claims 1-11, wherein steps (c) and (d) comprise culturing the Schwann cells in a container having a laminin-coated surface.

13. The method of claim 12, further comprising, directly following step (c), determining the percentage of Schwann cells in the culture and, if the percentage is less than 80%, transferring the Schwann cells to an uncoated container for a period of time to allow fibroblast adhesion to the container.

14. The method of any one of claims 1-13, wherein steps (c) and (d) comprise culturing the Schwann cells for a period of time to achieve 60%-90% confluence.

15. The method of claim 14, wherein steps (c) and (d) comprise culturing the Schwann cells for a period of time to achieve 80%-90% confluence.

16. The method of any one of claims 1-15, step (c) comprises culturing the primed Schwann cells at an initial P0 density of 13,333 cells/cm² for a period of time to achieve no greater than 90% confluence.

17. The method of any one of claims 1-16, wherein step (d) is performed for three passages.

18. The method of any one of claims 1-17, further comprising (f) washing the population of Schwann cells at least two times.

19. A method of producing a population of human Schwann cells, the method comprising
(a) incubating human fascicles with forskolin and heregulin for eight days to produce primed Schwann cells,
(b) incubating the primed Schwann cells with collagenase and neutral protease for 18 hours,
(c) preparing a suspension of primed Schwann cells in laminin-coated tissue culture containers at a density of 10,000 cells/cm² to 15,000 cells/cm²,
(d) culturing the Schwann cells until 80%-90% confluence,
(e) passaging the Schwann cells into larger laminin-coated tissue culture containers at an initial passage density of 6667 cells/cm² to 13333 cells/cm²,
(f) passaging the Schwann cells when 80%-90% confluence is obtained no more than three times, wherein the Schwann cells are seeded at an initial passage density of 6667 cells/cm² to 13333 cells/cm² at each passage,
(g) harvesting the population of human Schwann cells, and
(h) washing the harvested population of human Schwann cells at least twice.

20. An isolated population of Schwann cells obtained by the method of any one of claims 1-17.

21. A method of treating nerve injury in a subject, the method comprising administering to a subject in need thereof a population of Schwann cells obtained by the method of any one of claims 1-17.
